(19) United States
(12) Patent Application Publication
(10) Pub. No.: US 2011/0203174 A1
(43) Pub. Date: Aug. 25, 2011

(54) METHOD AND APPARATUS FOR EXTRACTING CARBON DIOXIDE FROM AIR

(76) Inventor: Klaus S. Lackner, Dobbs Ferry, NY (US)

(21) Appl. No.: 13/058,812

(22) PCT Filed: Aug. 11, 2009
(86) PCT No.: PCT/US09/53450

§ 371 Date: May 10, 2011

Related U.S. Application Data
(60) Provisional application No. 61/087,980, filed on Aug. 11, 2008.

(51) Int. Cl. A01G 1/00; B01D 53/14; F28F 13/00; G01N 33/00

(52) U.S. Cl. 47/58.1 R; 95/236; 96/234; 95/183; 95/156; 165/121; 73/23.2

(57) ABSTRACT

A method and apparatus for extracting CO₂ from air, and for delivering that extracted CO₂ to controlled environments, such as a greenhouse, or to open-air agricultural fields. The present disclosure allows the delivery of CO₂ to be made at times of highest demand. The present disclosure contemplates several geometric configurations to enhance the CO₂ extraction process. The present disclosure also provides a method of delivering the CO₂ to the controlled environment in response to demand, such as for example, by using a secondary sorbent as a buffer to store extracted CO₂.

The present disclosure in one aspect relates to removal of selected gases from air. The disclosure has particular utility for the extraction of carbon dioxide (CO₂) from air and the creation of a CO₂ enriched atmosphere and will be described in connection with such utilities, although other utilities are contemplated.

There is compelling evidence to suggest that there is a strong correlation between the sharply increasing levels of atmospheric CO₂ with a commensurate increase in global surface temperatures. This effect is commonly known as Global Warming. Of the various sources of the CO₂ emissions, there are a vast number of small, widely distributed emitters that are impractical to mitigate at the source. Additionally, large scale emitters such as hydrocarbon-fueled power plants are not fully protected from exhausting CO₂ into the atmosphere. Combined, these major sources, as well as others, have lead to the creation of a sharply increasing rate of atmospheric CO₂ concentration. Until all emitters are corrected at their source, other technologies are required to capture the increasing, albeit relatively low, background levels of atmospheric CO₂. Efforts are underway to augment existing emissions reducing technologies as well as the development of new and novel techniques for the direct capture of ambient CO₂. These efforts require methodologies to manage the resulting concentrated waste streams of CO₂ in such a manner as to prevent its reintroduction to the atmosphere.

The production of CO₂ occurs in a variety of industrial applications such as the generation of electricity power plants from coal and in the use of hydrocarbons that are typically the main components of fuels that are combusted in combustion devices, such as engines. Exhaust gas discharged from such combustion devices contains CO₂ gas, which at present is simply released to the atmosphere. However, as greenhouse gas concerns mount, CO₂ emissions from all sources will have to be curtailed. For mobile sources the best option is likely to be the collection of CO₂ directly from the air rather than from the mobile combustion device in a car or an airplane. The advantage of removing CO₂ from air is that it eliminates the need for storing CO₂ on the mobile device. Another advantage of removing CO₂ from the air is that it can be done at the site of CO₂ storage and that one can eliminate the need for long distance transport of CO₂.

Extracting carbon dioxide (CO₂) from ambient air would make it possible to use carbon-based fuels and deal with the associated greenhouse gas emissions after the fact. Since CO₂ is neither poisonous nor harmful in parts per million quantities, but creates environmental problems simply by accumulating in the atmosphere, it is possible to remove CO₂ from air in order to compensate for equally sized emissions elsewhere and at different times.

Most prior art methods, however, result in the inefficient capture of CO₂ from air because these processes heat or cool the air, or change the pressure of the air by substantial amounts. As a result, the net reduction in CO₂ is negligible as the cleaning process may introduce CO₂ into the atmosphere as a byproduct of the generation of electricity used to power the process.

Various methods and apparatus have been developed for removing CO₂ from air. For example, we have recently disclosed methods for efficiently extracting carbon dioxide (CO₂) from ambient air using capture solvents that either physically or chemically bind and remove CO₂ from the air. A class of practical CO₂ capture sorbents include strongly alkaline hydroxide solutions such as, for example, sodium or potassium hydroxide, or a carbonate solution such as, for example, sodium or potassium carbonate brine. See for example published PCT Application PCT/US05/29979 and PCT/US06/029238.

In co-pending U.S. application Ser. No. 11/866,326, filed Mar. 8, 2007, U.S. Publication No. U.S.-2008-0087165-A1, assigned to a common assignee, there are described a method and apparatus for extracting CO₂ from ambient air and for delivering that extracted CO₂ to a greenhouse or other controlled environment. The apparatus includes of a set of mobile air filters, comprised of a sorbent material with a strong humidity function, that is to say, an ion exchange resin having the ability to take up CO₂ as humidity is decreased, and give up CO₂ as humidity is increased. The filters are arranged to be moved into a collector system, where the filters are in the flow path of an air stream or other gas stream. The means of moving the filters in an out of the air stream may be, for example, a series of louvers or some type of track system. Once the filters have been sufficiently loaded with CO₂ they are exposed to high levels of moisture to release the CO₂ and regenerate the filters. This could be accomplished by wetting the filters with liquid water or, preferably, by exposing the filters to water vapor, for example, by exposing the filters to the humid atmosphere of a greenhouse. The partial pressure of the water vapor controls the equilibrium partial pressure of the CO₂ released. The water vapor pressure is in turn controlled by the temperature of the regeneration chamber. Typical temperatures range from 30° C. to 50° C.

Where the unit is designed to create CO₂ enriched air, the transformation occurs in the presence of air. Alternatively, where the object is to obtain concentrated CO₂, it may be necessary to remove, at least in part, the air from the chamber prior to adding moisture that stimulates the release of the CO₂. See for example PCT Application No. PCT/US08/60672, filed Apr. 17, 2008, incorporated by reference herein.

The result is a moist stream of CO₂ enriched air, where the rate of CO₂ production is driven by the ambient conditions and the size of the apparatus. In applications where the demand for CO₂ is also flexible the rate of CO₂ production is not necessarily matched to the immediate CO₂ demand.

This design produces CO₂ enriched air in such large volumes that it preferably will be consumed essentially immediately, thereby reducing or eliminating the need for on-site or off-site storage. A greenhouse, however, will have a time varying CO₂ demand that will vary with insolation, temperature, humidity, and the size of the plants inside. While it is may be possible to throttle the production of CO₂, it is in general not possible to substantively accelerate production past a design point, which suggests that the capital cost of the apparatus can be far larger for a device that has a strongly time varying demand, as is the case, for example, with a greenhouse. The prior art solution would therefore require sizing the unit for the maximum demand. Thus, there remains a need for an efficient and less costly configuration for CO₂ capture and delivery to a controlled environment, in particular, one with a varying demand for CO₂.

The present disclosure provides a system, i.e. a method and apparatus for extracting carbon dioxide (CO₂) from ambient air and for delivering that extracted CO₂ to a controlled environment. In broad concept, the present disclosure provides several options for improving the efficiency of a CO₂ collection system.

In one aspect of the present disclosure, a system is provided for collecting CO₂ and delivering the extracted CO₂ to a controlled environment wherein the filters are arranged in various geometric configurations designed for airflow and temperature control.

Another aspect of the present disclosure is directed to the conservation of heat in a controlled environment where there is a wide swing between day time and night time temperatures. The present disclosure is comprised of at least two reservoirs of a fluid, such as water, for storing heat. This aspect of the present disclosure is particularly useful when used in connection with a CO₂ collection system.

The present disclosure in another aspect provides a CO₂ buffer system that is operated with, or as part of, an air collector producing CO₂ enriched air for delivery to a controlled environment. The present disclosure will allow the CO₂ capture system to operate on a continuous basis even though demand for the CO₂ could be highly intermittent or variable. In one example, a secondary sorbent is provided to serve as a buffer to release the CO₂ in times of increasing demand and restrict the release of CO₂ in times of decreasing demand. In another example, the apparatus includes a plurality of filters that may be stored while saturated or partially saturated with CO₂. The filters may be regenerated and release CO₂ as the demand requires.

The present disclosure also provides a system for delivering CO₂ enriched to the point of demand. Alternatively, this example is also contemplated for use with open environments in additional to closed, controlled environments.

Finally, the present disclosure is discussed below primarily as implemented with a greenhouse. However, the disclosure is also intended to apply to any application in which the goal is to generate CO₂ enriched air.

Further features and advantages of the present disclosure will be seen from the following detailed description, taken in conjunction with the accompanying drawings, wherein

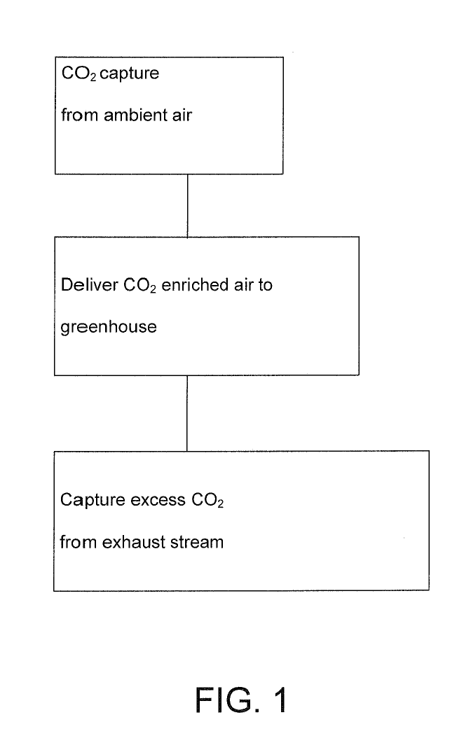

FIG. 1 is a flowchart describing a method for managing CO₂ in the operation of a greenhouse;

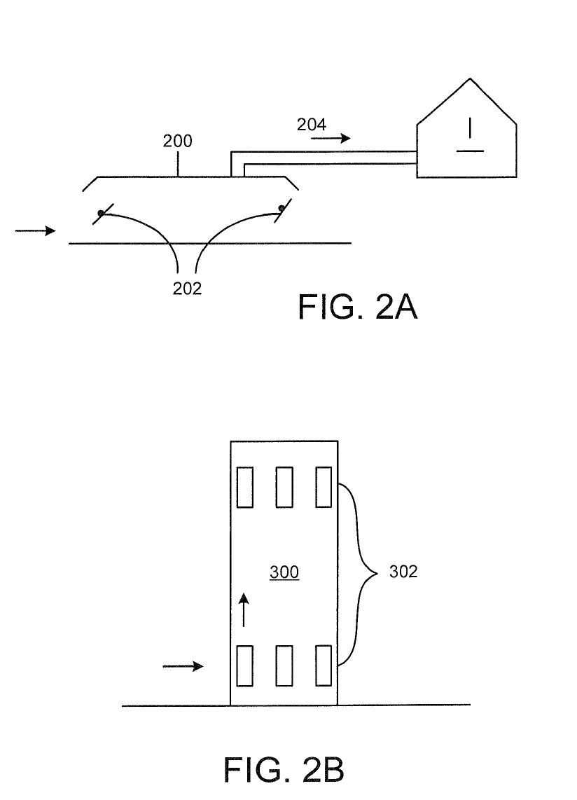

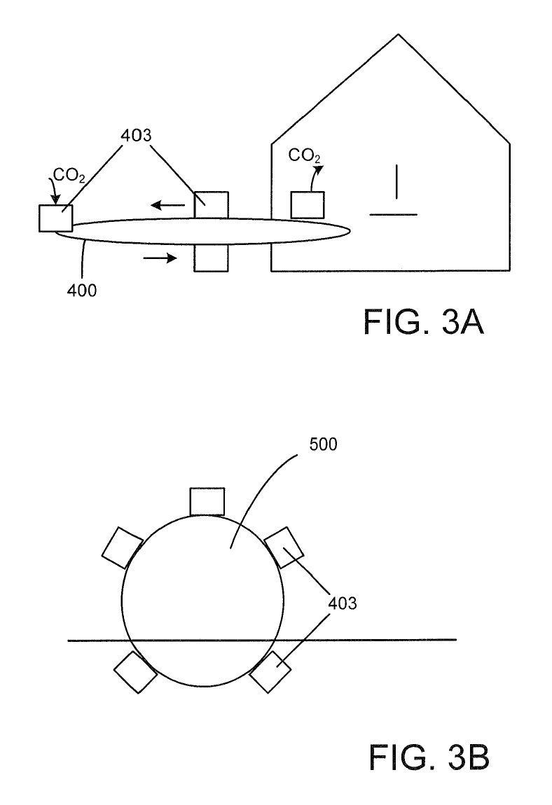

FIGS. 2A and 2B and FIGS. 3A and 3B are drawings of advantageous geometric configurations for the capture of CO₂ in accordance with the present disclosure;

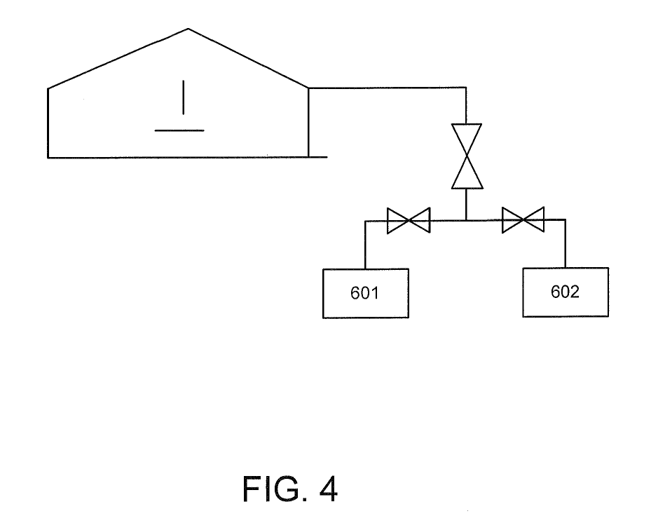

FIG. 4 is a schematic of an apparatus for managing heat in connection with a greenhouse;

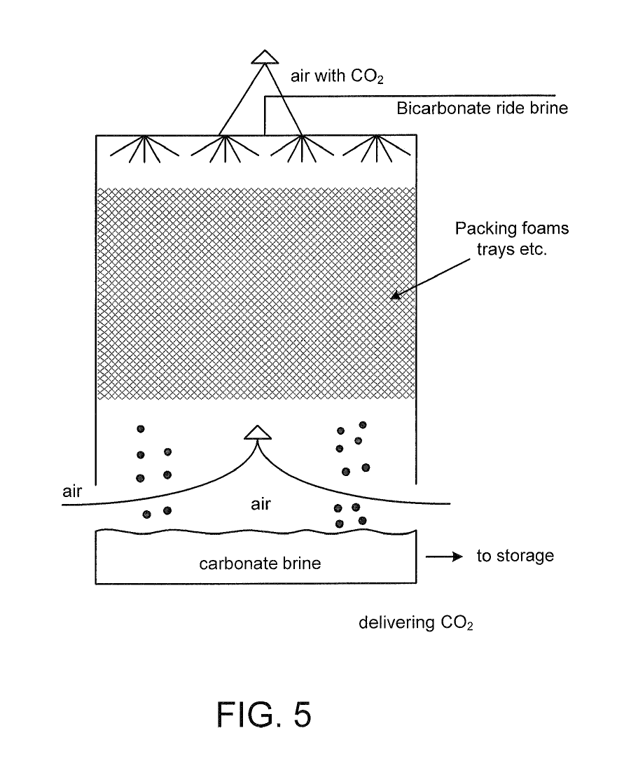

FIGS. 5-7 schematically illustrate CO₂ collection and delivery in accordance with various embodiments of the present disclosure; and

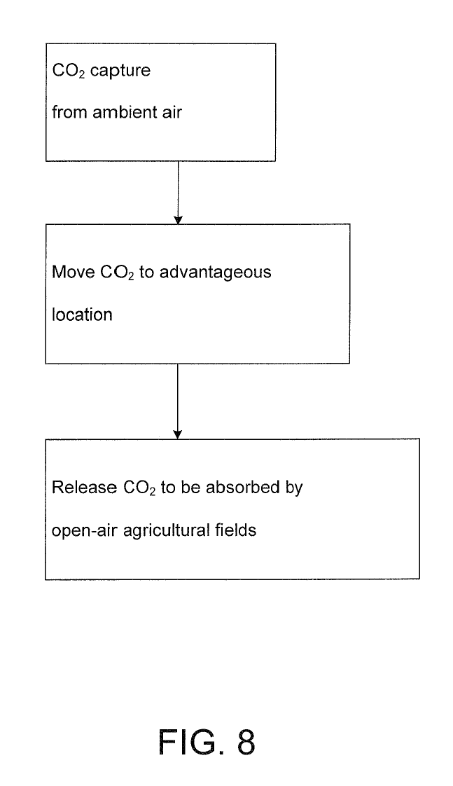

FIG. 8 is a flowchart describing a method for capturing CO₂ and delivering the CO₂ to an open-air field.

In co-pending U.S. application Ser. No. 11/866,326, filed Mar. 8, 2007, U.S. Publication No. U.S.-2008-0087165-A1, assigned to a common assignee, there are described a method and apparatus for extracting CO₂ from ambient air and for delivering the extracted CO₂ to a greenhouse or other controlled environment. The present disclosure provides several methods and systems for improving the efficiency of the method and apparatus described in the aforesaid application.

Air capture collectors utilizing a humidity swing work best in dry air. Under these conditions the equilibrium pressure of CO₂ above the sorbent (said pressure is a function of the loading state) is systematically lower. The determinative characteristic is best demonstrated by the absolute humidity. Hence cold air with high relative humidity for purposes of this discussion can be considered dry. In most greenhouse installations, air inside the greenhouse typically contains more moisture than ambient air outside the greenhouse. In general the humidity and temperature inside a greenhouse is relatively high. Where the humidity in the greenhouse air is much higher than that of the ambient air, the air inside the greenhouse can serve as the purge gas which drives the CO₂ off of the sorbent after it has been saturated with CO₂. Once the sorbent has released the bulk of its CO₂, it has been regenerated and will be returned to an outside air stream to collect additional CO₂. If the humidity levels between the inside and the outside are too close to one another to achieve a sufficient humidity swing, then it is necessary to wet the sorbent to force it to give up the collected CO₂. How this is achieved depends on the specific circumstances. For example, humid air, DI water, condensate, and pulses of steam are some of the ways disclosed in U.S. patent application Ser. No. 11/866,326 for wetting the resin. The following discussion relates primarily to the example just described, but should not be viewed as limited to this example as other resins and other controlled environments will also necessarily benefit from this disclosure and are contemplated by the present disclosure.

In sum, if the outside air is hotter than the greenhouse air, then one can create an air stream that is as hot as the outside air and fully saturated with water, without adding any energy input except for ambient heat. If it becomes necessary to raise the humidity level even higher, this can be accomplished either by heating the purge gas so that it can hold even more moisture, or, alternatively, by spraying water directly onto the sorbent. This procedure leads to a substantially complete release of CO₂ from the sorbent materials. In other words, the loading state changes from the bicarbonate form (one carbon atom per cation in the resin), to the carbonate form (one carbon atom per two cations in the resin). The carbonate state of the resin is considered the fully discharged form of the sorbent. One potential disadvantage of wetting the sorbent directly is that it will require quite additional time for the sorbent to dry in the outside air, thereby increasing the cycle time in the system.

The cycle time of the sorbent is an important parameter in assessing the performance of the system. The total capacity of the sorbent is fixed, and the total uptake rate per unit surface area is also relatively stable. Therefore a shorter cycle time leads to a reduction in the amount of sorbent necessary. Shorter cycle times are therefore one of the aims of the present disclosure.

An additional problem for enriching CO₂ in the greenhouse atmosphere arises if the greenhouse has to exhaust air to the outside, which may be the case, for example, if the greenhouse operation is limited in heat management. Thus, one may remove excess CO₂ from the exhaust stream to reduce the cost of CO₂ collection. See FIG. 1. This may be done by passing the greenhouse exhaust through a CO₂ collection system. Another potential source of CO₂ may be the CO₂ that is released through respiration inside the greenhouse at night.

In another aspect of the present disclosure, the collector system can take one of several advantageous geometric configurations. Referring to FIG. 2A, one example comprises a CO₂ extractor 200 that contains the resin material in a solid frame that forms a partial enclosure that is opened (or baffled 202) on two sides to let air in and out. The CO2 enriched air that results may be piped using piping 204 to the greenhouse 1.

Alternatively, one can provide a small tower 300 that at some level above ground contains a “sorbent filter” through which the air flows in a vertical path. See FIG. 2B. In this tower configuration, above and below the filter there are baffles 302 that can be opened and closed. In the closed system another set of connections will be opened to allow the volume to be exposed to a humid purge gas, which could be, for example, warm moist air from the greenhouse. The gas flows through the vertical tower because it is driven either by pressure differences between the top and bottom created by wind fields on the outside of the tower, (Bernoulli effect), or the tower may create an updraft or downdraft due to thermal convection. In one example for driving flow through the tower is to use solar heat that impinges on the sides of the tower to heat the gas as it rises through the tower, causing the tower to act like a small solar chimney. In another example a reverse flow tower is provided by locating a water source below the filter, the evaporative cooling resulting in a downward draft in the tower. By limiting this technique to the purging step, it is possible to combine convection with humidification of the purge gas. A third example employs mechanical devices to fan or drive the air flow. All of these examples serve the purpose of bringing air in contact with the resin material.

Another advantageous geometric configuration provides horizontal containers that can be opened and closed in a manner as described in relation to the tower configuration, but wherein the flow occurs in a horizontal direction. While it is still possible to utilize fans as above, this design is particularly advantageous if the airflow is driven by wind using the Bernoulli effect.

The two geometric configurations described above are similar in that the air filters are stationary. The airflow patterns assist the system in performing the various steps. The advantage of such a system is a great degree of simplicity, but the disadvantage is a relatively high cost in the construction of the container. While the container may not be required to be completely air tight, it does require substantial structural strength.

Another geometric configuration employs a different approach, wherein the filters are as open as possible to ambient air and stand in the wind or even move in the air. Such filters would have to be moved into an enclosure before returning the absorbed CO₂. The advantage of this design is that it is easier to deal with unpredictable and small air flows. The system can be stored away and kept out of the wind, if wind speed becomes too great. Indeed it may be possible to run the system even on very windy days, if it is in a much more compact form.

The above geometric configurations are aimed at reducing the amount of time required to absorb CO₂ from the ambient air. The advantages of these configurations will be minimized where the amount of time required to release the CO₂ to the greenhouse or other controlled environment becomes longer than the amount of time required for absorption. A few examples of conditions where the present disclosure might be useful are discussed below.

The present disclosure in another aspect may be used where temperatures inside and outside the greenhouse are similar, the air on the outside of the greenhouse has very low relative humidity, and the air on the inside of the greenhouse has very high relative humidity. The sorbent material readily will absorb CO₂ from the ambient outside air until it reaches a level that is close to equilibrium with the outside air. When the sorbent is exposed to the humid air on the inside of the greenhouse, it releases a fraction of the CO₂ that it has absorbed, thereby raising the CO₂ content inside the greenhouse. CO₂ levels of 1500 to 1700 ppm and greater are achievable in this arrangement.

Experimental data show that the time it takes the resin to respond to a sudden change in humidity is very short. This is particularly true where the resin is not wetted using liquid water, which would have to evaporate before the resin can again absorb CO₂. Consequently, it is not necessary for the resin become completely saturated with CO₂ while drying on the outside. Instead, it is possible to expose the resin for a brief period to the dry air on the outside during which time the resin will absorb some CO₂ from the air. After that, the resin is exposed to moist air on the inside and will readily release excess CO₂ at a rate that is similar to the uptake rate experienced on the outside. In effect, the change in humidity level reverses the flow of CO₂ into and out of the resin.

At the more or less static loading level we consider, the equilibrium partial pressure of CO₂ on the inside and the outside of the greenhouse are above and below, respectively, the actual partial pressure values achieved. As a result one can use a relatively small amount of resin to carry a large amount of CO₂ from the outside to the inside in a short period of time. The amount of resin required is proportional to the anticipated cycle time. If one assumes that the resin is to be exposed for 1 minute, then a rate of 15 moles per minute (1 ton per day) would require somewhere between 20 and 200 kg of resin. The lower estimate of 20 kg would require a deep swing in loading, and the high number would limit the swing to less than 0.07 mole/kg, which is a tiny fraction of the total capacity. On the other hand, if we assume that uptake and release rates are around 20 μmol/m²s the total surface area required is on the order of 8000 m². This correlates to an average material layer of 1/40 of a millimeter. It is therefore practical for the sorbent material to be coated on some surfaces and the thinnest possible coat is achieved. Since it is quite reasonable to pack between 500 and 1000 m² into a cubic meter, the volume requirement of the device is quite reasonable for this application. The material could be presented in thin sheets, in structured packings, or in strands (akin to furnace filters). The goal would be to allow for different flow patterns that at one time expose the material to the outside and then to air flows coming from the inside of the greenhouse.

One example useful in these circumstances is a simple tower, with two baffles, one at the top, one at the bottom, that can be opened or closed. In addition there is a smaller connection to the greenhouse through a second set of pipes connecting the system. These pipes can also be opened or closed. It is possible to have a fan in the tower, but it may be that the fan can be replaced either by a differential wind pressure (using the Bernoulli effect) or through convection. Since photosynthesis will similarly vary with the available sunlight, solar driven convection may be the most energy efficient way of operating this system.

Another example uses simple lightweight boxes filled with sorbent material with the wind blowing through horizontally. See FIG. 3A. The boxes 403 are moveable on a track like structure 400 and enter a closed box where they are then exposed to air flow from the inside of the greenhouse 1. They can be exposed to the open wind, or can be embedded in another chamber that adds an air blower. This construction might be advisable in case the system would otherwise stall for lack of wind.

Referring to FIG. 3B, it is possible to arrange the boxes on a wheel 500 that stands like a Ferris wheel and moves around. The prevailing wind could aim the wheel axis in the direction of the wind. The top section of the wheel would be exposed to the open air, on the bottom it may use a fan to drive the air through the system and in another section exchange air with the greenhouse. The filters could be arranged like a paddlewheel, wherein the system would see relative air flow even if the air is nearly completely stagnated.

It is worth noting that the flow of gas into the greenhouse can be much slower than the flow of air during open air exposure as the system can achieve a much higher CO₂ loading in the purge gas. If the loading with CO₂ turns out to be too high it can be reduced by further dilution within the greenhouse.

Another example where the present disclosure might be useful is where the temperature on the outside is substantially lower than inside the greenhouse. In this case the air on the outside will very likely have a much lower level of absolute humidity, as the maximum absolute humidity is limited at low temperatures. In such case, the resin may be moved in and out of the greenhouse on a wheel. However, moving the resin in and out of the greenhouse will increase heat losses from the greenhouse to the outside as the resin is being exposed to repeated warming and cooling cycles, though generally these heat losses will be small compared to the heat losses experienced by the greenhouse generally.

Accordingly, ignoring the heat losses as mentioned above, each of the options outlined above provide an improvement over the prior art. In some circumstances, however, it is possible that condensation will form on the resin as it enters into the warm moist chamber. Where this occurs, the total mass of resin required should be adjusted to reflect the actual speed of the cycle as limited by the effects of condensation.

While condensation may allow a quick release of CO₂, it may also impede the overall speed of the sorbent cycle and thus be detrimental. There are several ways to overcome the condensation problem according to the present disclosure. One option is to preheat outside air to warm up the resin. The heated air can be used to provide heat to the interior of the greenhouse. Further, if the heating process involves combustion of carbonaceous materials, the CO₂ produced can be use to enhance the CO₂ delivery system. It may or may not make sense to absorb this combustion-produced CO₂ onto the resin as well, depending the specific application.

The heat demand can be reduced by recovering some of the heat from the resin as it leaves the interior of the greenhouse. The heat exchange may not only involve air, but a heat transfer medium such as water, that is used to provide input and output heat. One or more heat reservoirs containing the medium can be arranged with a heat exchanger to carry the medium between the high temperature in the greenhouse and the relatively low temperature outside air. Each reservoir receives heat through the heat exchanger by cooling the unloaded resin. Each reservoir will provide heat through the heat exchanger for fully-loaded resin entering the greenhouse.

Unless there is carbon free source of heat available, the system may be allowed to shut off at some low outside temperature, as the heat provided for running the greenhouse will generate enough CO₂. As a rough measure, a 20° C. temperature difference between inside and outside will require approximately 20 kJ per mole of CO₂ bound in order to heat the resin up from the lower outside temperatures to the higher inside temperature. If the swing in the CO₂ is relatively small, such as where only 10% of the amount of CO₂ bound to the resin, the heat demand per mole of CO₂ could reach 200 kJ per mole without heat recovery. However, total losses from the greenhouse through the glass could be much larger than that. Thus, when the system is cold, there may be no need for additional CO₂. Solar heat may be an alternative source of heat in some applications and thus reopen the need for CO₂ augmentation. The availability of CO₂ thus makes the use of solar energy more interesting.

As an additional example, CO₂ can be recovered from the exhaust air of the greenhouse after removing excess water. Consider for example a greenhouse operating near or below freezing conditions, with the help of burners. In such instances, there is ample CO₂ available for plant growth, and it may be possible to collect some of the CO₂ from the exhaust air. The exhaust air may be run through a heat exchange loop, which first lets the air cool, and then reheats it once more before it lets the air escape. This can be viewed of as a mechanical equivalent of “penguin feet” for recapture of water and residual CO₂ during night time operations of a greenhouse. (Penguins conserve body heat by transferring heat from arterial blood flowing to the feet to venous blood returning from the feet, thereby eliminating much of the heat losses that would otherwise occur in the feet that are exposed to cold exterior temperatures.)

The warm air exiting the greenhouse is sent through a counter-stream heat exchanger where the air cools and water condenses out of the air. On the way back the air is reheated using the heat of condensation of the air being cooled. Water is recovered in this manner and the dry air is essentially reheated to the temperature of the greenhouse. At this point a CO₂ collection device as described above can be used in connection with other elements of the present disclosure to recover excess CO₂, to be used at an advantageous time, such as when heaters are not running.

This example may be used to recover the CO₂ from heaters that are positioned inside the greenhouse and which during maximum heating periods produce excess CO₂. This example may also be used to recover night time CO₂ from plant and soil respiration in the greenhouse which is not matched by CO₂ absorption through photosynthesis. This application has particular utility for operation in a desert environment where night time temperatures can drop very low relative to day time temperatures.

Reducing night time CO₂ levels on the inside of the greenhouse may also be beneficial to controlling plant growth. The approaches discussed above provides for controlling night time CO₂ inside the greenhouse with minimal nighttime venting. It is further possible to return the air after the water has been condensed out back to the inside of the greenhouse. This allows for additional water management in the greenhouse.

Another example of this concept could be in agricultural situations where animals are kept close by greenhouses. The present disclosure provides a transfer mechanism from one CO₂ producing enclosure to another enclosure where it is consumed. In this aspect of the disclosure, the air is dried with a water sorbent, and the water is returned after the CO₂ collection back into the stream. This method handles interactions between two systems of similar moisture level. This example could provide a way of lowering the moisture level inside the greenhouse, if so desired, without bringing in cold air.

Another example considers conditions where the air outside the greenhouse is dry, but substantially warmer than the air inside the greenhouse. In this case the CO₂ swing will likely depend on the difference in absolute humidity. If the swing is still sufficiently large to allow efficient operation, the greenhouse gas air may be used to regenerate the resin. If, however, the air is not humid enough to cause the resin to release its CO₂, it may be necessary to produce warmer air inside a chamber attached to the greenhouse into which the resins are brought. A small amount of air from the greenhouse is drawn into the chamber, and the high temperature outside raises the temperature and humidity inside this chamber. The amount of water that will need to be evaporated is still relatively small, and no additional heat is required if the system settles at a chamber temperature at or below ambient temperatures.

The air may be cooled before it is brought back into the greenhouse with an evaporative cooling system, forcing the condensation of some of the water on the inside of the greenhouse. It may be advantageous under these circumstances to drive the CO₂ content of the moist air as high as possible, because the amount of water involved will depend more on the amount of air used than on the amount of CO₂ freed. To accomplish this, the system may include a chamber that can raise the humidity of the controlled environment at ambient outside temperatures. It also is possible to run at even higher temperatures taking advantage of available solar heat. Under these conditions it even may be possible to run the system at such times when the outside air is hot and humid, wherein the system can create conditions of even higher temperatures and humidity levels.

In each of the above situations it is possible that the greenhouse demand for heating and cooling will be much greater than the demands of the system for heating and cooling. For example, plants in a greenhouse covering a hectare will consume about 0.2 moles of CO₂ per second. Producing the same amount of CO₂ from natural gas would provide 160 kW of heat, or 16 W per square meter. This is very small compared to the solar flux which is being absorbed in the greenhouse. During a day we would produce 17,000 moles of CO₂ or 15 MMBTU of heat. However, in most climates, a greenhouse needs to shed heat during the day rather than absorb it.

Therefore, the greenhouse or other controlled environment may be provided with a heat management system for a desert location of a greenhouse 1 where nights are cold and days are hot. Referring to FIG. 4, the heat management system is comprised of two reservoirs 601, 602, such as for example, underground aquifers or storage tanks that may be above ground or sunk into the ground. The reservoirs preferably are thermally insulated. A first reservoir 601 is maintained at an elevated temperature, comparable to the high temperature in the day. The second reservoir 602 is maintained at a low temperature essentially to match night temperatures, or perhaps even lower. In this manner, the two reservoirs serve as a thermal swing mass inside the greenhouse. By way of example, during the day the water can be pumped from the second reservoir to cool the greenhouse as the water absorbs heat. At night, the water from the first storage system is pumped to maintain a temperature inside the greenhouse higher than the ambient temperature outside. Exposing the water to ambient air will cool the water even further. Evaporative cooling also may be used.

Assuming that all the solar heat ends up heating the greenhouse, one will need about 200 liters of water to absorb enough heat to raise the waters temperature by 20° C., in order to absorb all the sunshine's energy that hits one square meter (17 MJ). Similarly, one would then have a lot of heat available to keep the greenhouse warm at night. For a 1 ha (hectare) installation, one would have to contain 2000 m³ of water. A tank 10 meter deep having a radius of about 8 m can hold approximately 2000 m³. This is substantially larger than the CO₂ collector system described above. The use of eutectics may reduce the amount of storage space required for the reservoirs. Alternatively, it is possible to use a large gravel bed through which the water percolates. One bed is cooled at night while the other is heated during the day. For the greenhouse example specifically, using evaporation and condensation as a means of heat transport would be advantageous.

In another aspect, the present disclosure provides a method and apparatus for extracting CO₂ from ambient air and for delivering that extracted CO₂ to a greenhouse or other controlled environment, such as described in co-pending U.S. application Ser. No. 11/866,326, co-owned and incorporated by reference herein, and further comprising a secondary sorbent that can act as a buffer in the system to allow delivery of the CO₂ when needed. The main purpose for the secondary sorbent is to create a buffer between the collector and the consumer. There are many potential secondary sorbents, but an optimal secondary sorbent is one that undergoes a large load swing in the range of CO₂ concentrations that are optimal for the application. In the greenhouse example of the disclosure, the desirable range is between 0.1% and 10% of CO₂ in the off stream. A particularly preferred range would be between 0.3 and 3% of CO₂ in the off stream.

Potentially effective secondary sorbents include, but are not limited to solid and liquid amines (particularly weak based amines), zeolites, or other physical sorbents. Nano-engineered sorbents, such as for example the metal-organic frameworks developed by Omar Yaghi at UCLA, could provide another option. The optimal sorbent undergoes its most rapid variation in loading near the point of operation. As the atmosphere increases in CO₂ concentrations, the system will fill up with CO₂. As it decreases, the material will give most of it back. In these designs the air acts as a carrier gas that brings the CO₂ stream in contact with the secondary sorbent for additional loading and that is brought in contact with CO₂ depleted air in order to add CO₂ from the buffer to the air.

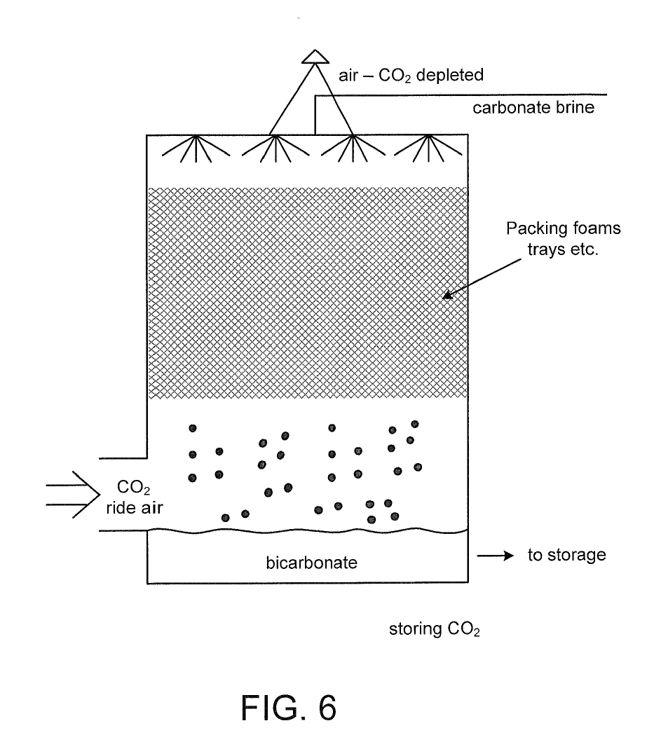

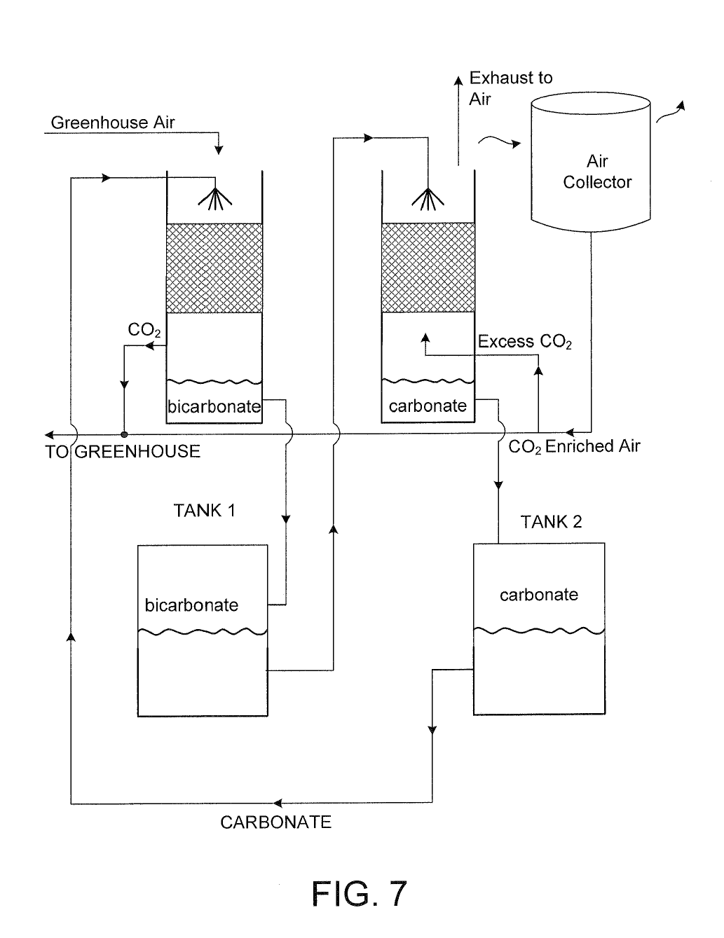

Liquid sorbents are particularly useful, as one can utilize very standard gas-liquid interfaces for absorption and release, using standard packed beds or trays. It is easy to store the liquid in a large container that is put into proximity of the air capture device. Preferably, there will be at least two containers: one with CO₂ saturated fluid and one with fluid that is ready to absorb additional CO₂. FIG. 5 shows a buffer container where CO₂ rich air is passed upwardly through the packed beds or trays, over which a carbonate brine is caused to flow. The carbonate brine accepts much of the CO₂, becoming a bicarbonate, and is stored. FIG. 6 shows the similar container as it releases the CO₂. In this instance air is passed upward through the packed beds or trays over which the bicarbonate brine is caused to flow. The bicarbonate brine releases CO₂, enriching the air stream. It also is possible to have a plurality of such liquid containers where each container represents a different loading state of the sorbent. FIG. 7 shows the system as a whole, including distribution and storage.

Other configurations are also possible. For example, the secondary sorbent, such as a carbonate brine, may be used to regenerate the CO₂ collection filters directly. In this example, the CO₂ collection filters do not necessarily need to be comprised of a sorbent with a significant humidity function.

One example of a simple buffer sorbent is a carbonate/bicarbonate brine that has been loaded with CO₂ to a desired concentration. This desired concentration preferably is at a few percent of CO₂. By passing off-gas from the regenerator through the stripped buffer fluid, it is possible to capture most of the CO₂ that has been released. If instead CO₂ depleted air is directly brought in contact with loaded sorbent then the sorbent will impart CO₂ to the offstream.

In the greenhouse implementation, the system typically will load the brine with CO₂ during dark hours and will use the brine to augment the CO₂ delivery during daylight hours. The optimal transfer of CO₂ can be achieved by adjusting the concentration of the brine and/or the temperature of the brine. The level of loading in relation to temperature can be shown using Harte's model, which calculates the equilibrium for sodium carbonate-bicarbonate solutions at a temperature and partial pressure of CO₂ (see Harte et al., “Absorption of Carbon Dioxide in Sodium Carbonate-Bicarbonate Solutions,” Industrial and Engineering Chemistry, vol. 25, no. 5, 528-531 (1986)):

(X²C^{1.29})/(SP(1−X)(185−t)=10

where X represents the fraction of the total sodium in the solution, C represents the sodium normality of the solution, S represents the solubility of CO₂ in water at a given temperature, P represents the partial pressure of CO₂ expressed in atmospheres, and t represents temperature in Celsius. A first calculation using Harte's model, suggests that a 3% loading at 35° C., is a good level at which to operate the system. However, it nevertheless is possible to exploit a wide range of parameters.

One method of operation is to make the CO₂ buffer an add-on to the air collector. The collector creates a CO₂ enriched gas stream, which is either passed directly to the greenhouse or is passed through a secondary sorbent chamber where CO₂ is removed from the gas stream. In this manner the CO₂ content of the exhaust is reduced when not all of the CO₂ is needed. When the CO₂ demand exceeds what the air capture device can deliver, some of the input air is passed directly over the secondary sorbent system in order to collect CO₂. Here, much like the design in our previous application, PCT/US08/60672, one can arrange several chambers in series to create a counter-stream system in which the most depleted sorbent is exposed to the air with the lowest CO₂ content. Such a counter-stream system is very useful for loading the secondary sorbent with CO₂ and is also useful for releasing CO₂ from the sorbent into the offgas stream. In either case, a counter-streaming arrangement makes it possible to increase the size of the loading swing of the buffer sorbent.

It also may be useful to direct available heat toward the sorbent releasing CO₂ to enhance the release process, while cooling might be performed in the system (e.g. by evaporative cooling) prior to removing CO₂ from the gas stream, and would have the effect of conditioning the secondary sorbent to not impart of CO₂.

In this manner it is possible to collect CO₂ on a 24-hour basis and even take advantage of the higher concentration of CO₂ inside the greenhouse at night to reload the storage buffer. This reloading in principle could be accomplished with a secondary sorbent, but in practice it may be the air from the greenhouse that is run through a standard air collector system such as described in our several prior applications listed in Appendix A. During peak demand during the day, the CO₂ stored on the secondary sorbent will be released into the greenhouse. The swing may be amplified by taking advantage of the temperature difference between day and night.

Using a brine as a secondary sorbent, the apparatus may operate with a swing of about 0.1 mol/liter, which appears easily achievable based on Harte's model mentioned infra. This would suggest that a large tank of liquid with approximately 15 cubic meter of solution would be required for a typical application. This is not excessive in view of the size of the greenhouse, or the size of the collector. Condensation water from the greenhouse can be used as make-up water.

Another method of implementing the buffer is to use the carbonate brine directly to wash the resin. The advantage of this method would be a faster transfer from the resin to the brine, but the disadvantage of this method is a higher water consumption. Thus, the particular conditions will dictate which example is more desirable.

It also is possible to use a carbonate brine as a direct interface to the greenhouse. For example, it would be possible to install a number of packed beds inside a greenhouse through which interior greenhouse air is routed in order to pick up CO₂ from a percolating brine. Rather than pumping CO₂ rich air through the greenhouse, the air collector would deliver a bicarbonate rich brine, which is transformed back into a carbonate brine as it delivers its CO₂ to the greenhouse.

In another aspect of the present disclosure, the goal of delivering CO₂ to the controlled environment, such as for example a greenhouse, is accomplished by including additional resin filters that may be loaded with CO₂ and stored for later release. One disadvantage of this example is the cost of the resin filters. For example, in our present design there are two sets of filters. At any one time, one set is loading or collecting while the second set unloading or regenerating. Loading a set takes about one hour, while unloading takes another hour. Hence to cover five hours of collection would require another four sets of resin filters, effectively tripling the number of resin pads inside the system. One may be able to gain a little more than five hours by overloading the resins during the times the system would otherwise stay idle, thus reducing the need for additional sets of filters. This approach may make sense, if for example these filters are discharged by bringing them inside a greenhouse.

It is worth noting in connection with this example, that the regeneration units should be designed to keep up with the maximum demand. Still, regeneration utilizing humid air within a greenhouse is quite simple and does not add much cost. Furthermore, all other design considerations suggest lowering the buffering capacity of the resin, a development that will become a greater problem as filters are stored for periods of time.

Another aspect of the present disclosure may be used to improve the yield of crops grown in open fields. Many farming crops could sustain increased growth rates if the CO₂ level in the ambient air around the plants could be increased. Rapid growth on a field can lead to a local suppression in the CO₂ level at least near ground level. The air capture devices described above can be used to collect CO₂ from a source in the vicinity of the field, on nearby fields that are lying idle, or on land that is not in agricultural use and deliver the collected CO₂ to the growing crops.

One example of the present disclosure provides collector devices, portable or stationary, that are deployed in locations where a slight reduction in CO₂ is acceptable, or where CO₂ is in abundance, and after absorbing CO₂ the CO₂ laden collector material is treated to release the collected CO₂ at a site adjacent to the field, and regenerated. It is thus possible to let high CO₂ levels “waft” over the field, or alternatively pipe the air through tubes, that distribute the high concentration CO₂ near the ground thereby engulfing the plants into elevated levels of CO₂. See FIG. 8.

The present disclosure could be deployed in any number of ways for various applications. However, the discussion here is focused on an example based on an ion exchange resins that can release CO₂ when exposed to water.

There are various methods and systems for transporting the CO₂ to the edge of the field. One method is to transport the saturated resin. The designs described above which place the material into a “box” configuration are extremely well suited to that. The box can be exposed to high humidity by either adding water, or by pumping small amounts of humidified air into the box, causing the resin to release the CO₂.

In arid areas that perform agriculture, water is usually available as irrigation water. One alternative example of the idea would be to expose the boxes to sunshine, creating a slight convective current in the box and having the box draw in air over a wetted filter, which will dramatically raise the humidity on the inside of the box.

Another example involves pumping air through the box and into pipes which distribute the CO₂ throughout the field. In this case, one simply humidifies the air prior to pumping it through the resin container. Another option that can be considered, if the available water is sufficiently clean, is to directly wet the resin with the water and thus create a thermal flow in the box which carries high levels of CO₂.

It is further possible, particularly in orchards, to put CO₂ collectors near the ground, which will collect CO₂ at night when the resin is dry and the absolute humidity is low. During the day, when the temperature is high and irrigation is turned on, the units become wet and in response will exhale CO₂ collected during dry times. It is an advantageous feature of this example that the CO₂ is released when moisture is present, which increases the rate of photosynthesis. In such a design the sorbent layers should be sufficiently thick in order to obtain cycle times which approach a full day.

The present disclosure also provides a method for determining the amount of fossil carbon that has been incorporated into a controlled environment, such as a greenhouse, by measuring carbon-14 content. Where fossil fuels are concerned, these materials have been kept away from the atmosphere for millions of years and all traces of carbon-14 isotopes that are found in surface materials will have long decayed.

Plants that have been grown with air captured CO₂ on the other hand will reflect this fact in a normal level of carbon-14, as the carbon-14 from the fossil-fuel-produced CO₂ will be readily present in the plant. Hence it is possible to use a carbon-14 detection system to determine the amount of “fossil” carbon that has been incorporated into the plant versus the amount of modern, i.e. biomass or atmospheric CO₂. This may in turn be used to determine, e.g., carbon credits.

A greenhouse gas operation in a cold climate that relies in part on natural gas to create heat and in part on air captured CO₂ to satisfy its carbon balance can prove by this method that its accounting of CO₂ from different sources is indeed correct.

The accounting of CO₂ becomes more complex if the input stream involves waste carbon that is to be burned. Again the carbon-14 content can be used to complete the accounting. In another application of this disclosure, a carbon-14 inventory of the flue gases leaving a waste-to-energy plant can tell immediately how much of the fuel has been based on fossil carbon and how much on modern (biomass) carbon.

There are many methods for measuring carbon-14 known in the art, and that can be used to determine the carbon-12 to carbon-14 ratio in vegetable matter, algae matter, in CO₂ effluents, in other materials that have incorporated carbon from different sources, and thus determine accurately the ratio of fossil carbon to surface carbon that is incorporated in this device.

The purpose of this aspect of the disclosure is to account for carbon sources incorporated into a material in a continuous fashion and to provide a simple tool for verifying claims of air capture advocates, and/or as a way of determining carbon credits. If CO₂ is taken from the air, it will have a very similar C-14 contribution to the CO₂ in the air. If the CO₂ output has been stretched with fossil CO₂ then the carbon-14 ratio will change.

It should be emphasized that the above-described embodiments of the present device and process, particularly, and “preferred” embodiments, are merely possible examples of implementations and merely set forth for a clear understanding of the principles of the disclosure. Many different embodiments of the method and apparatus for extracting carbon dioxide from air described herein may be designed and/or fabricated without departing from the spirit and scope of the disclosure. All these and other such modifications and variations are intended to be included herein within the scope of this disclosure and protected by the following claims. Therefore the scope of the disclosure is not intended to be limited except as indicated in the appended claims.

APPENDIX A
GLOBAL US APPLICATIONS:

| Ser. No. | Date Filed |
|---|---|
| 11/346,522 | Feb. 2, 2006 |
| 60/649,341 | Feb. 2, 2005 |
| 60/703,098 | Jul. 28, 2005 |
| 60/703,099 | Jul. 28, 2005 |
| 60/703,100 | Jul. 28, 2005 |
| 60/703,097 | Jul. 28, 2005 |
| 60/704,791 | Aug. 2, 2005 |
| 60/728,120 | Oct. 19, 2005 |
| 60/780,466 | Mar. 08, 2006 |
| 11/683,824 | Mar. 8, 2007 |
| 60/780,467 | Mar. 8, 2006 |
| 60/827,849 | Oct. 2, 2006 |
| 11/866,326 | Oct. 2, 2007 |
| 60/829,376 | Oct. 13, 2006 |
| 60/866,020 | Nov. 15, 2006 |
| 60/912,649 | Apr. 18, 2007 |
| 60/912,379 | Apr. 17, 2007 |
| 60/946,954 | Jun. 28, 2007 |
| 60/985,596 | Nov. 5, 2007 |
| 60/980,412 | Oct. 16, 2007 |
| 60/985,586 | Nov. 5, 2007 |
| 60/989,405 | Nov. 20, 2007 |
| 61/029,831 | Feb. 19, 2008 |
| 61/080,110 | Jul. 11, 2008 |
| 61/058,876 | Jun. 4, 2008 |
| 61/058,881 | Jun. 4, 2008 |
| 61/058,879 | Jun. 4, 2008 |
| 61/074,972 | Jun. 23, 2008 |
| 61/074,976 | Jun. 23, 2008 |
| 61/080,630 | Jul. 14, 2008 |
| 61/079,776 | Jul. 10, 2008 |

1. A process for removing carbon dioxide from air, comprising passing ambient air in contact with a sorbent to absorb carbon dioxide from the air, delivering the carbon dioxide to a controlled environment, and removing excess carbon dioxide from an exhaust stream exiting the controlled environment.

2. The process of claim 1, wherein removing excess carbon dioxide from an exhaust stream includes passing the exhaust through a heat exchange loop, the heat exchange loop comprising cooling the exhaust to condense moisture from the exhaust in a first part of the loop, using heat from the first part of the loop to reheat the dry exhaust in a second part of the loop, and bringing the exhaust in contact with a sorbent to absorb carbon dioxide from the exhaust.

3. An apparatus for adding carbon dioxide to a controlled environment, comprising an extractor for extracting carbon dioxide from ambient air outside of the controlled environment and delivering the carbon dioxide into the controlled environment, wherein the extractor further comprises an ion exchange material in a solid frame that forms a partial enclosure having at least two openings to allow air to enter and exit the extractor.

4. The apparatus of claim 3, wherein the frame comprises a plurality of horizontal containers having baffled openings at each end.

5. The apparatus of claim 3, wherein the solid frame comprises a tower, and wherein the at least two openings include baffles, at least one of the openings being located in an upper portion of the tower and at least one of the openings is located in a lower portion of the tower, the ion exchange material being located between said upper portion and said lower portion.

6. The apparatus of claim 5, wherein air flow through the tower is characterized by one of the following:
(a) wherein the air flow through the tower is driven in an upward vertical direction by pressure differences between the upper and lower portions of the tower;
(b) wherein air flow through the tower is driven in an upward vertical direction by solar heat which impinges on the sides of the tower to heat the air as it rises through the tower;
(c) wherein moisture is added to the air having an evaporative cooling effect, and thereby driving the air in a downward vertical direction; and
wherein the tower includes one or more fans for driving the air through the tower.

7. The apparatus of claim 5, wherein the tower is connected to the controlled environment by a set of pipes.

8. An apparatus for adding carbon dioxide to a controlled environment which comprises an extractor for extracting carbon dioxide from air outside of the controlled environment and delivering the extracted carbon dioxide into the controlled environment, wherein the extractor includes a plurality of moveable filters comprised of a carbon dioxide capture material that are placed in contact with ambient air to capture carbon dioxide and moved on a track into an enclosure to release the extracted carbon dioxide.

9. The apparatus of claim 8, wherein the air inside the controlled environment has a greater absolute humidity than the air outside the controlled environment, and further including a device for moving the moveable filters into or adjacent to the controlled environment to release carbon dioxide into the controlled environment.

10. The apparatus of claim 8, wherein the moveable filters are attached to the track.

11. The apparatus of claim 8, wherein the filters are placed on a moveable wheel that turns with the prevailing wind to optimize the flow of ambient air over the filters.

12. A method for delivering carbon dioxide to a controlled environment, comprising capturing carbon dioxide from ambient air using a plurality of moveable filters, the moveable filters having a strong humidity function; storing the filters until needed; and exposing the moveable filters to warm, humid air of the controlled environment to release the carbon dioxide when desired.

13. A process for removing carbon dioxide from ambient air and for delivering carbon dioxide to a controlled environment wherein the temperature of the ambient air is substantially lower than the air within the controlled environment to which the carbon dioxide is to be delivered, comprising the steps of heating the ambient air, passing the heated air in contact with a sorbent to absorb carbon dioxide from the air, and delivering the carbon dioxide to a controlled environment.

14. The process of claim 13, wherein the carbon dioxide is delivered to the controlled environment by placing the sorbent in contact within the controlled environment, whereupon the sorbent releases the carbon dioxide as a result of a humidity swing.

15. The process of claim 13, further comprising the step of removing excess carbon dioxide from an exhaust stream exiting the controlled environment.

16. The process of claim 14, further comprising recovering some of the heat from the resin as it leaves the controlled environment.

17. An apparatus for managing heat in a controlled environment without producing excess carbon dioxide, comprising at least two thermally insulated reservoirs located adjacent to the controlled environment, wherein a first reservoir is maintained at an elevated temperature and a second reservoir is maintained at a lower temperature.

18. The apparatus of claim 17, wherein the first reservoir is maintained at or near a temperature comparable to the day time high temperature of ambient air, and wherein the second reservoir is maintained at or near a temperature comparable to the night time low temperature of ambient air.

19. The apparatus of claim 17, further comprising one or more pipes for carrying a heat exchange fluid between the reservoirs, and at least one pump for circulating the heat exchange fluid in the pipes.

20. The apparatus of claim 17, wherein the reservoirs contain either water or a eutectic solution.

21. A process for capturing carbon dioxide and delivering the captured carbon dioxide to a controlled environment, comprising the steps of capturing carbon dioxide from the air of a livestock facility and delivering the captured carbon dioxide to a controlled environment.

22. A method for managing a carbon dioxide level in a controlled environment, comprising using a primary sorbent to collect carbon dioxide, transferring at least a part of the collected carbon dioxide to a secondary sorbent, storing the collected carbon dioxide in the secondary sorbent, and releasing the stored carbon dioxide as desired for operation of the controlled environment.

23. The method of claim 22, wherein the secondary sorbent undergoes a load swing depending on a concentration of carbon dioxide.

24. The method of claim 23, wherein the secondary sorbent undergoes a load swing between carbon dioxide concentrations of 0.1% and 10%.

25. The method of claim 22, wherein the secondary sorbent is selected from a group consisting of: a carbonate brine, a liquid amine, a zeolite, activated carbon, and a non-engineered sorbent.

26. The method of claim 22, wherein the secondary sorbent is used to regenerate the primary sorbent directly.

27. The method of claim 22, wherein the secondary sorbent is maintained near 35° C.

28. The method of claim 22, wherein the carbon dioxide is transferred directly from the primary sorbent to the controlled environment at times of highest demand.

29. The method of claim 22, wherein the secondary sorbent is heated to aid in release of carbon dioxide to the controlled environment.

30. An apparatus for managing the level of carbon dioxide in a controlled environment, comprising: a primary sorbent for capturing carbon dioxide from an air stream; an enclosure in which carbon dioxide from the primary sorbent may be released; at least two gas-liquid interfaces for recapturing the carbon dioxide on a secondary sorbent; and at least one container for storing the secondary sorbent.

31. A method for carbon dioxide fertilization of open agricultural fields, comprising capturing carbon dioxide from air adjacent the field and releasing the carbon dioxide in a manner that will raise the carbon dioxide concentration near the plants in the field.

32. The method of claim 31, wherein the capture of carbon dioxide is captured at a time when the plants on the field are not photosynthetically active.

33. The method of claim 31, wherein the carbon dioxide is captured downwind from the field to be fertilized.

34. The method of claim 33, wherein the collectors are moved upwind prior to be transformed into carbon dioxide releasing units.

35. The method of claim 34, where the collectors are installed on a track.

36. The method of claim 34 where the collectors are truck mounted.

37. The method of claim 33, wherein the collectors use the captured carbon dioxide to enrich a gas stream that is pumped upstream and released at locations in the field that can be optimized with regard to carbon dioxide retention in the field and carbon dioxide exposure of the plants.

38. The method of claim 31, where the capture medium is sensitive to a humidity or moisture swing and releases carbon dioxide if brought in contact with excess moisture.

39. The method of claim 38, where humidity or moisture is provided from the field's irrigation supply.

40. The method of claim 38, where the humidity or moisture is provided from stored rain water.

41. The method of claim 38, wherein recovery from the humidity swing is accelerated using solar heat to dry the capture medium.

42. The method of claim 31, where the capture medium is heat sensitive and carbon dioxide is released by exposing the material to elevated temperatures.

43. The method of claim 42, wherein the temperature swing is partially or completely brought about with solar heat that increases the temperature during the release cycle.

44. The method of claim 42, wherein the temperature swing is at least partially brought about by evaporative cooling during an uptake phase of the collecting unit.

45. The method of claim 31, wherein the carbon dioxide release is accomplished at the capture site and the carbon dioxide enriched gas is pumped upstream of the field prior to its distribution.

46. A method for determining the amount of fossil carbon that has been incorporated into a controlled environment containing plants, comprising measuring the carbon-14 content of the plants.

47. The method of claim 46, wherein the controlled environment is a greenhouse.

48. The method of claim 47, wherein the atmosphere in the greenhouse is enriched at least in part with carbon dioxide from the burning of fossil fuels.

49. The method of claim 48, wherein the atmosphere is further enriched with carbon dioxide from an air capture device.
